(19) United States
(12) Patent Application Publication
(10) Pub. No.: US 2026/0259208 A1
(43) Pub. Date: Sep. 3, 2026

(54) Anti-Vimentin Antibodies are Associated With Higher Severity of Sjogren's Syndrome

(71) Applicant: Oklahoma Medical Research Foundation, Oklahoma City, OK (US)

(72) Inventor: Umesh Deshmukh, Edmond, OK (US)

(21) Appl. No.: 19/117,533

(22) PCT Filed: Oct. 4, 2023
(86) PCT No.: PCT/US2023/075952

§ 371 Date: Apr. 1, 2025

Related U.S. Application Data
(60) Provisional application No. 63/413,705, filed on Oct. 6, 2022.

(51) Int. Cl. G01N 33/564; C07K 14/47

(52) U.S. Cl. CPC G01N 33/564; C07K 14/47; G01N 2333/47; G01N 2800/101; G01N 2800/52; G01N 2800/56

(57) ABSTRACT

A method, assay, and kit for detecting a biomarker comprising: obtaining or having obtained a biological sample from the subject suffering from Sjogren's syndrome; and assaying a biological sample obtained from the subject for the presence of anti-vimentin antibodies, wherein vimentin used in the assay is a post-translationally modified soluble vimentin, wherein the biological sample is selected from the group consisting of tears, saliva, salivary gland tissue, blood, serum, and plasma; and wherein the anti-vimentin autoantibodies are detected using an assay selected from at least one of: multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

### CROSS-REFERENCE TO RELATED APPLICATIONS

This application is a non-provisional patent application of and claims priority to U.S. provisional patent application Ser. No. 63/413,705 filed on Oct. 6, 2023, the contents of which are incorporated by reference in its entirety.

### TECHNICAL FIELD OF THE INVENTION

The present invention relates in general to the field of Sjögren's Syndrome (also known as Sjögren's disease), and more particularly to autoantibody biomarkers Sjögren's Syndrome.

### STATEMENT OF FEDERALLY FUNDED RESEARCH

None.

### BACKGROUND OF THE INVENTION

Without limiting the scope of the invention, its background is described in connection with Sjögren's syndrome.

Sjögren's syndrome (SS)/Sjögren's Disease (SjD) is a rheumatic autoimmune disease selectively targeting salivary and lacrimal glands, leading to painful dry mouth and eyes, oral infections, severe dental caries/tooth loss, fatigue, arthritis, nervous system involvement and malignant B cell lymphoma. Current internationally accepted disease classification criteria rely on either the presence of anti-Ro antibodies (these may target either the Ro60 antigen, Ro52 antigen or both) or the presence of focal lymphocytic infiltrates in a minor salivary gland lip biopsy for diagnosis.

Despite these advances, novel biomarkers are needed to identify Sjögren's syndrome patients at risk for a higher severity of disease, e.g., reduced fluid secretion that manifests into the classic dry mouth and dry eye symptoms of the disease.

### SUMMARY OF THE INVENTION

As embodied and broadly described herein, an aspect of the present disclosure relates to an assay for detecting a biomarker comprising: obtaining or having obtained a biological sample from the subject suffering from Sjögren's syndrome; and assaying the biological sample obtained from the subject to determine an amount of anti-vimentin antibodies; wherein the amount of anti-vimentin autoantibodies is detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection; and if the patient has a higher amount of anti-vimentin autoantibodies when compared to a healthy control or an average of patients with moderate to low symptoms of SS, identifying that the patient has a higher severity Sjögren's syndrome. In one aspect, the biological sample is selected from a saliva, a blood, a plasma, a serum, or a tear sample. In another aspect, the presence of anti-vimentin antibodies is indicative of a higher severity Sjögren's syndrome. In another aspect, the higher severity Sjögren's syndrome is oral dryness, ocular dryness, or both.

As embodied and broadly described herein, an aspect of the present disclosure relates to a method of detecting and treating one or more symptoms of Sjögren's Syndrome in a subject comprising: obtaining or having obtained a biological sample from the subject; detecting an amount of anti-vimentin antibodies in the biological sample; and administering one or more therapeutic compositions selected from the group consisting of saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome if the amount of anti-vimentin antibodies in the sample is higher than in a healthy control or an average of patients with moderate to low symptoms of Sjögren's syndrome. In one aspect, the anti-vimentin antibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the biological sample is selected from a saliva, a blood, a plasma, a serum, or a tear sample. In another aspect, the higher amount of anti-vimentin antibodies when compared to an amount in a healthy control or an average of patients with moderate to low symptoms of SS is indicative of a higher severity Sjögren's syndrome. In another aspect, the higher severity Sjögren's syndrome is oral dryness, ocular dryness, or both.

As embodied and broadly described herein, an aspect of the present disclosure relates to an assay for detecting a higher severity Sjögren's syndrome consisting essentially of: assaying a biological sample obtained from the subject suffering from one or more symptoms of Sjögren's syndrome for an amount of anti-vimentin autoantibodies, wherein the biological sample is selected from the group consisting of tears, saliva, salivary gland tissue, blood, serum, and plasma; wherein the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection, and wherein if the patient has a higher amount of anti-vimentin autoantibodies when compared to a healthy control or an average of patients with moderate to low symptoms of SS, identifying that the patient has a higher severity Sjögren's syndrome. In one aspect, the biological sample is selected from a saliva, a blood, a plasma, a serum, or a tear sample. In another aspect, the higher severity Sjögren's syndrome is oral dryness, ocular dryness, or both.

As embodied and broadly described herein, an aspect of the present disclosure relates to a kit comprising: a synthetic or recombinant peptide or protein, covalently attached to a solid support or a detectable label, wherein the synthetic or recombinant peptide or protein is vimentin; and instructions for detecting an amount of anti-vimentin antibodies in a biological sample from a patient suspected of having Sjögren's Syndrome and comparing to an amount of anti-vimentin antibodies in a healthy control or an average of patients with moderate to low symptoms of Sjögren's syndrome. In another aspect, the solid support is selected from the group consisting of a multiwell plate, an enzyme-linked immunosorbent assay (ELISA) plate, a microarray, a bead, a porous strip, and a nitrocellulose filter. In another aspect, the kit is an assay selected from the group consisting of a multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the kit further comprises a secondary antibody labeled directly or indirectly with a detectable moiety. In another aspect, if the amount of anti-vimentin antibodies in a sample is higher than an amount in a healthy control or an average of patients with moderate to low symptoms of SS, the higher amount of anti-vimentin antibodies is indicative of higher severity Sjögren's syndrome determined by oral and ocular dryness.

As embodied and broadly described herein, an aspect of the present disclosure relates to a method of detecting a biomarker comprising: obtaining a biological sample from the patient suspected of having Sjögren's Syndrome (SS); detecting or having detected an amount of anti-vimentin autoantibodies in the biological sample; comparing the amount of anti-vimentin autoantibodies in the biological sample to an amount in a healthy control or an average of patients with moderate to low symptoms of SS; and determining, by a computer device, that the patient has a higher amount of anti-vimentin autoantibodies when compared to the healthy control or patients with moderate to low symptoms of SS; and if the patient has a higher amount of anti-vimentin autoantibodies identifying that the patient has a higher severity Sjögren's syndrome. In one aspect, the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the biological sample is selected from a saliva, a blood, a plasma, a serum, or a tear sample. In another aspect, the method further comprises the step of treating the patient with at least one of: saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B shows an analysis of anti-vimentin antibody levels in SS patients and their association with clinical features of the disease. FIG. 1A is a graph that shows the sera from SS patients, non-SS sicca patients (Non-SS), and healthy controls (HC) were analyzed for reactivity to vimentin. Results are displayed as OD at 490 nm. Statistical significance was determined by the Kruskal-Wallis test, followed by Dunn's multiple comparison test. A p<0.05 was considered significant. The dotted line shows an arbitrary cut-off of mean OD+2SD HC and non-SS sicca samples. All samples above this cut-off were considered positive; the frequency is indicated below the figure. Statistical significance is determined by the Chi-square test. FIG. 1B shows that patients were stratified as positive (Pos) or negative (Neg) for different clinical parameters and levels of anti-vimentin antibodies compared between the 2 groups. Statistical significance was determined by the Mann-Whitney test, and a p<0.05 was considered significant. Note the most significant (p<0.0001) association was seen with saliva flow (WUSF). None of the samples tested were only positive for anti-La. The anti-Ro/La pos group includes 20 anti-Ro positive samples but negative for anti-La.

FIG. 2 shows that the O.D._{490 nm} readings were significantly higher when Baculovirus (BV)-expressed protein was used as a substrate for coating the ELISA plates.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Abbreviations: ANA: Anti-nuclear antibody, EMT: Epithelial to mesenchymal transition, MCV: Mutated citrullinated vimentin, OSS: Ocular surface staining score, RF: Rheumatoid factor, SS: Sjögren's Syndrome, vBS: van Bijsterveld score, WUSF: Whole unstimulated saliva flow.

Sjögren's Syndrome, also known as Sjögren's disease (SjD), is a rheumatic autoimmune disease selectively targeting salivary and lacrimal glands, leading to painful dry mouth and eyes, oral infections, severe dental caries/tooth loss, fatigue, arthritis, nervous system involvement, and malignant B cell lymphoma. Current internationally accepted disease classification criteria rely on either the presence of anti-Ro antibodies (these may target either the Ro60 antigen, Ro52 antigen, or both), or the presence of focal lymphocytic infiltrates in a salivary gland lip biopsy for diagnosis. Either one of these features, in combination with one or more objective dryness measures, is necessary for the fulfillment of classification criteria for SS.

Vimentin is a 57 kDa Type III intermediate filament protein, expressed in multiple tissues and cell types, but particularly at higher levels in enteric glia cells, adipocytes, fibroblasts, endothelial cells, and macrophages [1]. Vimentin plays a crucial role in diverse cellular functions involving adhesion, migration, differentiation, proliferation, and invasion [reviewed in 2]. In several rheumatic disorders, vimentin is a target of autoimmune response, and anti-vimentin antibodies have evolved as an important diagnostic and prognostic biomarker for some of these disorders [3].

Sjogren's Syndrome (SS) is a chronic autoimmune rheumatic disorder affecting multiple organ systems [4]. Immune-mediated injury of the exocrine salivary and lacrimal glands leads to reduced fluid secretion, which manifests into the classic dry mouth and dry eye symptoms of the disease. Autoantibodies reactive with vimentin, particularly the mutated citrullinated form of vimentin (MCV), have been detected in the sera of SS patients [5-10]. The MCV used in immunoassays has amino acid Gly at positions 19 and 59 instead of Arg. The recombinant protein made in E. coli is purified and citrullinated in vitro with peptidyl-arginine deaminase to generate MCV [11]. While this form of vimentin is present in synovial fluid from RA patients [12] and was the rationale for screening sera from RA patients, its existence in SS is not established. Furthermore, several RA studies investigating anti-MCV antibodies, sera from other rheumatic autoimmune disorders, including SS patients, were included for comparison or as controls. Hence, the role of anti-vimentin antibodies in SS pathogenesis has not been carefully studied. To address these issues, the inventors investigated anti-vimentin antibody responses in a well-characterized cohort of SS patients in this study. In addition, the inventors evaluated their association with multiple clinical features of the disease. This study demonstrates that anti-vimentin antibody response in SS is associated with higher disease severity.

As used herein, the term “biomarker” or “biomarkers” refer to one or more characteristics that are objectively measured and evaluated as indicators of a normal or abnormal biological process, pathogenic (disease) processes, or pharmacologic responses to therapeutic interventions. As used in the context of Sjögren's syndrome patients, the biomarkers are auto-antibodies that target certain proteins as shown herein.

As used herein, the terms “detectable”, “detectable biomarkers”, or “detectable labels” are used interchangeably to refer to directly or indirectly detecting a compound or composition that is conjugated directly or indirectly to the composition to be detected, e.g., a protein, element, or other molecule, such as an antibody or enzyme to generate a “labeled” composition. Detectable compounds and/or elements can be detected due to their specific functional properties and/or chemical characteristics, the use of which allows the agent to which they are attached or attachable to be detected, and/or further quantified if desired, such as, e.g., an enzyme, radioisotope, electron dense particles, magnetic particles or chromophore. There are many types of detectable labels, including fluorescent labels, which are easily handled, inexpensive and nontoxic. The detectable portion can be attached to, e.g., an antibody that is specific for human antibodies, such that it forms a sandwich with the antigens, e.g., a sandwich ELISA or other secondary binding of agents to one or more detectable labels.

As used herein, the term “treating” refers to curing as well as ameliorating at least one symptom of Sjögren's syndrome. Treatment for Sjögren's syndrome may include saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome.

As used herein, the term “effective amount” refers to the amount of a compound or agent administered or delivered to the patient which is most likely to result in the desired treatment outcome. The amount is empirically determined by the patient's clinical parameters including, but not limited to the stage of disease, age, gender, histology, and likelihood for recurrence.

As used herein, the phrase “a post-translationally modified vimentin” refers to a vimentin that has been recombinantly produced in a host cell that adds post-translational modification to the vimentin. The inventors found that vimentin manufactured in bacteria, which did not fold correctly and which do not add post-translational modifications to the protein, failed to provide sufficient signal when trying to detect anti-vimentin antibodies from human serum. By way of explanation, but in no way a limitation of the present invention, it may be the result of antibodies that are directed to conformational epitopes that rely on correct folding of the protein and antigenic epitopes that include the posttranslational modifications in the context of vimentin, rather than the vimentin itself. It was found that, surprisingly, soluble vimentin produced in host cells that post-translationally modified the vimentin led to the identification and quantitation of the anti-vimentin antibodies in human serum from patients with Sjögren's syndrome. As such, the present invention encompasses soluble and post-translationally modified vimentin, which is a vimentin manufactured in host cells such as mammalian cells, insect cells, plant cells, fungal cells, or other cells that post-translationally modify vimentin to more closely mimic human vimentin.

Patients: All experiments were in accordance with the Helsinki Declaration and were approved by the Oklahoma Medical Research Foundation (OMRF) Institutional Review Board. Banked serum samples from 204 individuals [primary SS (n=122), non-SS sicca (n=48), healthy individuals (n=34)] were obtained from the biorepository of the Oklahoma Sjögren's syndrome Center of Research Translation (OSSCORT). The clinical data on demographics, van Bijsterveld score (vBS), ocular surface staining score (OSS), Schirmer's test (mm/5 min), whole unstimulated saliva flow (WUSF; ml/15 minutes), minor salivary gland biopsy (Focus Score), anti-Ro/La, Rheumatoid Factor (RF) and Anti-Nuclear Antibody (ANA) was obtained from the OSSCORT database and are shown in Table 2. The patient cohort and data collection methods have been previously described in detail [13]. The diagnosis of primary SS and non-SS Sicca was based on the American College of Rheumatology (ACR) and the European League Against Rheumatism (EULAR) classification criteria [14].

Detection of antibody to Vimentin: An ELISA-based assay was used to determine antibodies to purified recombinant human vimentin generated in a baculovirus expression system (Sino Biologicals, Wayne, PA, USA) as previously described [15]. Briefly, vimentin in bicarbonate buffer (5 μg/ml) was coated on ELISA plates overnight at 4° C. All sera were tested at a 1:100 dilution. In each plate, serial dilutions of pooled serum samples positive for anti-vimentin were used as calibrators. Bound antibody was detected with HRP-conjugated goat anti-human IgG (Southern Biotech, Birmingham, AL, USA). O-phenylenediamine (Sigma Aldrich, St. Louis, MO, USA) was used as a substrate to determine enzyme activity, and the reaction was stopped with 2.5N sulfuric acid. The results are presented as absorbance at 490 nm. The cut-off for anti-Vimentin positivity was set at mean±2SD of the non-SS sicca and healthy control sera.

Importantly, it was found that the use of recombinant E. coli-produced vimentin provided an insufficient signal in the ELISA assay. The reactivity of human serum samples was compared between purified full-length recombinant Vimentin expressed in insect cells with Baculovirus Expression System (BV) versus Vimentin expressed in E. coli (EC). The EC Vimentin had 98% amino acid sequence homology with the BV Vimentin.

ELISA plates were coated with BV-expressed human Vimentin diluted in carb-bicarb buffer, pH 9.5 (5 μg/mL, 100 μL/well, giving a final concentration of 500 ng/well) and with EC-expressed mouse vimentin diluted in 8M Urea buffer, pH 8.0 (10 μg/mL, 100 μL/well giving a final concentration of 1000 ng/well). Please note there is a 98% homology in human and mouse protein sequences. The EC-expressed Vimentin had to be coated in Urea buffer as it is insoluble in Carb-bicarb buffer. The plates were kept overnight at 4° C.

The next day, the plates were washed 3 times with PBS and blocked with 1% BSA diluted in PBS (200 μL/well). After 1 h incubation at room temperature, plates were washed with PBS containing 0.05% Tween-20 (PBST). Human serum samples were diluted (1:100) in PBST containing 1% BSA and added to ELISA plates (100 μL/well), followed by a 2 h incubation at room temperature. Plates were washed 5 times with PBST. This was followed by adding HRP-conjugated Goat anti-human IgG diluted in PBST containing 1% BSA (1:4000, dilution, 100 μL/well). After 1 h incubation at room temperature, plates were washed 5 times, and color developed using Sigma Fast OPD substrate following manufacturer's instructions. The color reaction was stopped by adding 2.5N sulfuric acid (50 μL/well), and plates were read in an ELISA plate reader at 490 nm. Each serum sample was run in duplicates. Wells that did not get diluted human serum samples served as background control. The average O.D. of background control was subtracted from the average O.D. obtained for each human serum sample. The data was plotted using Graph Pad Prism software, and the Mann-Whitney test was used to determine statistical significance.

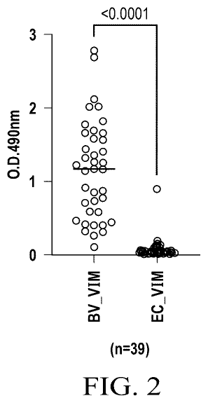

FIG. 2 shows that the O.D._{490 nm} readings were significantly higher when BV-expressed protein was used as a substrate for coating the ELISA plates. These data demonstrate that BV-expressed Vimentin is a superior substrate for detecting anti-Vimentin antibodies in human serum samples.

Statistical Methods: Prism 9.4 (GraphPad Software, LLC, San Diego, CA, USA) was used for all analyses. Normality tests were performed on each dataset, and non-parametric tests were used for non-Gaussian distributions. Mann-Whitney test was used to compare two populations, and the Kruskal-Wallis test, followed by Dunn's post-test for multiple comparisons, was used to compare three populations. Correlations were determined by Spearman's method, and the Chi-square test was used for contingency tables. The differences between groups with a p-value (two-tailed)<0.05 were considered significant.

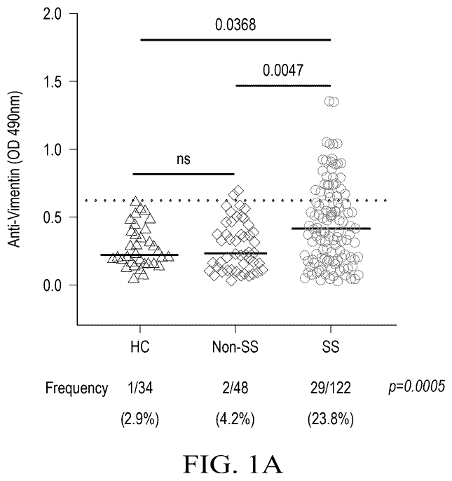

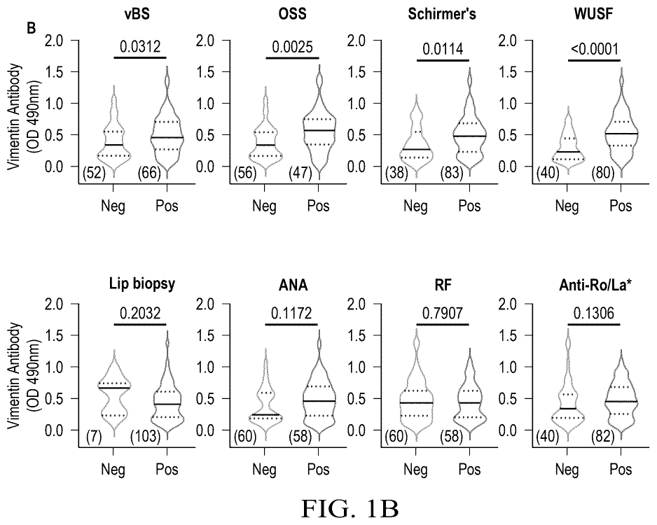

It is demonstrated herein that antibodies reactive with vimentin are readily detected in the sera of SS patients, and their presence is associated with higher severity of oral (saliva flow) and ocular (tear flow and corneal damage) features of the disease. Serum samples from patients with primary SS (n=122), non-SS sicca (n=48), and healthy individuals with verified absence of autoimmunity (n=34) were studied for anti-vimentin antibodies by ELISA (FIGS. 1A and 1B). A range of reactivity to vimentin was seen in all groups (FIG. 1A). However, SS patients (n=122) had significantly higher anti-vimentin IgG levels compared to healthy controls (n=34; p=0.037) and non-SS sicca (n=48; p=0.005). Furthermore, the frequency of anti-vimentin positivity (OD>cut-off) was significantly higher in SS patients compared to the two control groups (p=0.0005, Chi-square test).

Previous studies using serum samples from SS patients have reported the presence of circulating antibodies to vimentin and MCV [5-10]. However, the clinical relevance of anti-vimentin reactivity in SS has not been investigated. Thus, in this study, patients were stratified as positive or negative for each clinical criterion for oral (WUSF, lip biopsy score) and ocular (Schirmer's test for tear flow and vBS and OSS for corneal damage) features of the disease. The anti-vimentin antibody levels were compared between these two groups (FIG. 1). Reactivity to vimentin was significantly higher in patients positive for vBS (p=0.031), OSS (p=0.003), Schirmer's test (p=0.011), and WUSF (p<0.0001). Although anti-vimentin levels trended to be lower in patients positive for lip biopsy, they were not statistically significant.

Available quantitative data were used to determine correlations between different clinical criteria and anti-vimentin antibody levels (Table 1). Statistically significant positive correlations, albeit with moderate correlation coefficients, were seen between anti-vimentin levels and vBS and OSS. Anti-vimentin titers showed a negative correlation with WUSF. Of note was the lack of correlation between anti-vimentin levels and age. Overall, these results suggest that the immune response to vimentin is associated with higher disease severity, and it is not just an indicator of a non-specific, age-dependent autoimmune response.

TABLE 1
Correlation between anti-Vimentin antibody and
quantitative clinical criteria in SS patients.

| Anti-Vimentin | | 95% confidence | | XY pairs |
|---|---|---|---|---|
| versus | Spearman r | interval | p Value | (n) |
| Age | 0.1352 | −0.049 to 0.310 | 0.1377 | 122 |
| vBS | 0.2041 | 0.019 to 0.376 | 0.0266 | 118 |
| OSS | 0.2478 | 0.051 to 0.425 | 0.0116 | 103 |
| Schirmer's | −0.1564 | −0.331 to 0.029 | 0.0879 | 120 |
| WUSF | −0.3523 | −0.504 to −0.179 | <0.0001 | 120 |
| Focus Score | 0.1635 | −0.117 to 0.419 | 0.2375 | 54 |

SS patients harbor multiple autoantibody specificities, the prominent ones being ANA, anti-Ro/La, and RF [16]. In the patient cohort in this study, positivity for ANA, anti-Ro/La, and RF was 76%, 67%, and 49%, respectively. However, the levels of anti-vimentin antibodies were not significantly associated with positivity for these autoantibody specificities. Thus, these data suggest that anti-vimentin positivity is not just reflective of an elevated autoantibody response in SS patients.

TABLE 2
Characteristics of healthy volunteers, non-SS
Sicca and SS patients in this study

| | | Controls | | Non-SS Sicca | | SS | |
|---|---|---|---|---|---|---|---|
| Sex | n | 34 | | 48 | | 122 | |
| Females | n (%) | 29 | (85.3) | 45 | (93.8) | 113 | (92.6) |
| Race | n | 34 | | 48 | | 121 | |
| White | n (%) | 31 | (91.2) | 39 | (81.3) | 107 | (88.4) |
| Others* | n (%) | 3 | (8.8) | 8 | (16.7) | 14 | (11.6) |
| Age^{#} | median | 44 | | 56 | | 57 | |
| | IQR | 33-54 | | 47-67 | | 51-66 | |
| vBS | n | 28 | | 41 | | 118 | |
| positive | n (%) | 0 | (0) | 12 | (29.2) | 66 | (55.9) |
| OSS | n | 28 | | 33 | | 103 | |
| Positive | n (%) | 0 | (0) | 7 | (21.2) | 47 | (45.6) |
| Schirmer's | n | 34 | | 46 | | 121 | |
| Positive | n (%) | 0 | (0) | 14 | (30.4) | 83 | (68.6) |
| WUSF | n | 33 | | 46 | | 120 | |
| Positive | n (%) | 0 | (0) | 17 | (36.9) | 80 | (66.7) |
| Focus Score | n | 20 | | 38 | | 110 | |
| Positive | n (%) | 0 | (0) | 14 | (36.8) | 103 | (93.6) |
| Anti-Ro | n | 34 | | 48 | | 122 | |
| positive | n (%) | 0 | (0) | 2 | (4.17) | 82 | (67.2) |
| Anti-La | n | 34 | | 48 | | 122 | |
| positive | n (%) | 0 | (0) | 2 | (4.17) | 62 | (50.8) |

*Includes African American, American Indian, Asian, >1 race;
^{#}control vs sicca and control vs SS p < 0.001, sicca vs SS p = 0.757;

Autoimmune responses to vimentin have been noted in other autoimmune disorders. Contrary to the findings herein, a recent report suggested that anti-vimentin antibodies were more typical for secondary SS [17]. However, in that report, a peptide (YSLHNAGPWSLHQ)(SEQ ID NO:1) with only 25% homology with vimentin was used to screen patient serum samples, and reactivity to the whole vimentin protein was not investigated. Thus, the conclusion reached by the authors that primary SS patients lack anti-vimentin antibodies is inoperative. The present study used purified recombinant whole vimentin generated in the baculovirus expression system. Mammalian proteins generated in this expression system undergo post-translational modifications and closely resemble the native protein.

Why tolerance to vimentin, an abundantly present cytoskeletal protein, is abrogated in different autoimmune/inflammatory disorders is not clear. By way of explanation, but in no way a limitation of the present invention, it may be that certain unique properties of vimentin might render it to be immunogenic. Vimentin undergoes post-translational modifications (acetylation, carbamylation, glycosylation S-nitrosylation phosphorylation) at multiple residues. These post-translational modifications influence vimentin functions by modifying its structure and assembly of individual filaments. Furthermore, under inflammatory stimuli, vimentin can shuttle to the cellular surface and can also be secreted. Thus, the ready availability of this self-protein to the adaptive immune system under an inflammatory milieu can render it highly immunogenic.

These studies demonstrate that the presence of anti-vimentin in SS patients is associated with higher disease severity. Therefore, anti-vimentin antibodies are a novel biomarker for evaluating disease severity in SS.

As embodied and broadly described herein, an aspect of the present disclosure relates to an assay for detecting a biomarker comprising, consisting essentially of, or consisting of: obtaining or having obtained a biological sample from the subject suffering from Sjögren's syndrome; and assaying the biological sample obtained from the subject to determine an amount of anti-vimentin antibodies; wherein the amount of anti-vimentin autoantibodies is detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection; and if the patient has a higher amount of anti-vimentin autoantibodies when compared to a healthy control or an average of patients with moderate to low symptoms of SS, identifying that the patient has a higher severity Sjögren's syndrome. In one aspect, the biological sample is selected from a saliva, a blood, a plasma, a serum, or a tear sample. In another aspect, the presence of anti-vimentin antibodies is indicative of a higher severity Sjögren's syndrome. In another aspect, the higher severity Sjögren's syndrome is oral dryness, ocular dryness, or both.

As embodied and broadly described herein, an aspect of the present disclosure relates to a method of detecting and treating one or more symptoms of Sjögren's syndrome in a subject comprising, consisting essentially of, or consisting of: obtaining or having obtained a biological sample from the subject; detecting an amount of anti-vimentin antibodies in the biological sample; and administering one or more therapeutic compositions selected from the group consisting of saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome if the amount of anti-vimentin antibodies in the sample is higher than in a healthy control or an average of patients with moderate to low symptoms of Sjögren's syndrome. In one aspect, the anti-vimentin antibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the biological sample is selected from a saliva, a blood, a plasma, a serum, or a tear sample. In another aspect, the higher amount of anti-vimentin antibodies when compared to an amount in a healthy control or an average of patients with moderate to low symptoms of SS is indicative of a higher severity Sjögren's syndrome. In another aspect, the higher severity Sjögren's syndrome is oral dryness, ocular dryness, or both.

As embodied and broadly described herein, an aspect of the present disclosure relates to an assay for detecting a higher severity Sjögren's syndrome comprising, consisting essentially of, or consisting of: assaying a biological sample obtained from the subject suffering from one or more symptoms of Sjögren's syndrome for an amount of anti-vimentin autoantibodies, wherein the biological sample is selected from the group consisting of tears, saliva, salivary gland tissue, blood, serum, and plasma; wherein the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection, and wherein if the patient has a higher amount of anti-vimentin autoantibodies when compared to a healthy control or an average of patients with moderate to low symptoms of SS, identifying that the patient has a higher severity Sjögren's syndrome. In one aspect, the biological sample is selected from a saliva, a blood, a plasma, a serum, or a tear sample. In another aspect, the higher severity Sjögren's syndrome is oral dryness, ocular dryness, or both.

As embodied and broadly described herein, an aspect of the present disclosure relates to a kit comprising, consisting essentially of, or consisting of: a synthetic or recombinant peptide or protein, covalently attached to a solid support or a detectable label, wherein the synthetic or recombinant peptide or protein is vimentin; and instructions for detecting an amount of anti-vimentin antibodies in a biological sample from a patient suspected of having Sjögren's syndrome and comparing to an amount of anti-vimentin antibodies in a healthy control or an average of patients with moderate to low symptoms of Sjögren's syndrome. In another aspect, the solid support is selected from the group consisting of a multiwell plate, an enzyme-linked immunosorbent assay (ELISA) plate, a microarray, a bead, a porous strip, and a nitrocellulose filter. In another aspect, the kit is an assay selected from the group consisting of a multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the kit further comprises a secondary antibody labeled directly or indirectly with a detectable moiety. In another aspect, if the amount of anti-vimentin antibodies in a sample is higher than an amount in a healthy control or an average of patients with moderate to low symptoms of SS, the higher amount of anti-vimentin antibodies is indicative of higher severity Sjögren's syndrome determined by oral and ocular dryness.

As embodied and broadly described herein, an aspect of the present disclosure relates to a method of detecting a biomarker comprising, consisting essentially of, or consisting of: obtaining a biological sample from the patient suspected of having Sjögren's syndrome (SS); detecting or having detected an amount of anti-vimentin autoantibodies in the biological sample; comparing the amount of anti-vimentin autoantibodies in the biological sample to an amount in a healthy control or an average of patients with moderate to low symptoms of SS; and determining, by a computer device, that the patient has a higher amount of anti-vimentin autoantibodies when compared to the healthy control or patients with moderate to low symptoms of SS; and if the patient has a higher amount of anti-vimentin autoantibodies identifying that the patient has a higher severity Sjögren's syndrome. In one aspect, the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the biological sample is selected from a saliva, a blood, a plasma, a serum, or a tear sample. In another aspect, the method further comprises the step of treating the patient with at least one of: saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome.

A person of skill in the art would readily recognize that steps of various above-described methods can be performed by programmed computers. For example, using the computer it is possible to apply an algorithm to the amount of anti-vimentin antibodies, the algorithm generating a Sjögren's syndrome score based on a comparison of the measured protein expression levels to reference levels. The algorithm can also be the results of machine learning, for which non-limiting examples are selected from a machine learning algorithm, a clustering algorithm, a support vector machine, or combinations thereof. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as a magnetic disk and/or magnetic tape, hard drives, or optically readable digital data storage media. The embodiments are also intended to cover computers programmed to perform said steps of the above-described methods.

The functions of the various elements shown in the figures, including any functional blocks labelled as “modules”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

The Human Protein Atlas, Vimentin RNA tissue cell expression. www.proteinatlas.org/ENSG00000026025-VIM/tissue+cell+type (accessed 5 Oct. 2022).
Danielsson, F., Peterson, M. K., Caldeira Aranjo, H., Lautenschloger, F., & Gad, A. (2018). Vimentin Diversity in Health and Disease. Cells, 7(10), 147. doi.org/10.3390/cells7100147
Musaelyan, A., Lapin, S., Nazarov, V., Tkachenko, O., Gilburd, B., Mazing, A., Mikhailova, L., & Shoenfeld, Y. (2018). Vimentin as antigenic target in autoimmunity: A comprehensive review. Autoimmunity reviews, 17(9), 926-934. doi.org/10.1016/j.autrev.2018.04.004
Baldini, C., Arnaud, L., Aogin, T., Beretta, L., Bellocchi, C., Bouillot, C., Burmester, G. R., Cavagna, L., Cutolo, M., de Vries-Bouwstra, J. K., Doria, A., Ferro, F., Fonseca, J. E., Fonzetti, S., Fulvio, G., Galetti, I., Gottenberg, J. E., Hachulla, E., Krieg, T., La Rocca, G., A. Mariette, X. (2022). Sjogren's syndrome and other rare and complex connective tissue diseases: an intriguing liaison. Clinical and experimental rheumatology, 40 Suppl 134(5), 103-112. doi.org/10.55563/clinexprheumatol/3y0has
Kurki, P., Helve, T., & Virtanen, I. (1983). Antibodies to cytoplasmic intermediate filaments in rheumatic diseases. The Journal of rheumatology, 10(4), 558-562.
Sos, L., Szekanecz, Z., Szabo, Z., Fekete, A., Zeher, M., Horvath, I. F., Danke, K., Kapitny, A., Vogvori, A., Sipka, S., Szegedi, G., & Lakos, G. (2007). Clinical evaluation of anti-mutated citrullinated vimentin by ELISA in rheumatoid arthritis. The Journal of rheumatology, 34(8), 1658-1663.
Poulsom, H., & Charles, P. J. (2008). Antibodies to citrullinated vimentin are a specific and sensitive marker for the diagnosis of rheumatoid arthritis. Clinical reviews in allergy & immunology, 34(1), 4-10. doi.org/10.1007/s12016-007-8016-3
Nicaise Roland, P., Grootenboer Mignot, S., Bruns, A., Hurtado, M., Palazzo, E., Hayem, G., Dieudo, P., Meyer, O., & Chollet Martin, S. (2008). Antibodies to mutated citrullinated vimentin for diagnosing rheumatoid arthritis in anti-CCP-negative patients and for monitoring infliximab therapy. Arthritis research & therapy, 10(6), R142. doi.org/10.1186/ar2570
Mutlu, N., Bicakcigil, M., Tasan, D. A., Kaya, A., Yavuz, S., & Ozden, A. I. (2009). Comparative performance analysis of 4 different anti-citrullinated protein assays in the diagnosis of rheumatoid arthritis. The Journal of rheumatology, 36(3), 491-500. doi.org/10.3899/jrheum.080656
Liu, X., Jia, R., Zhao, J., & Li, Z. (2009). The role of anti-mutated citrullinated vimentin antibodies in the diagnosis of early rheumatoid arthritis. The Journal of rheumatology, 36(6), 1136-1142. doi.org/10.3899/jrheum.080796
Bang, H., Egerer, K., Gauliard, A., Lohke, K., Rudolph, P. E., Fredenhagen, G., Berg, W., Feist, E., & Burmester, G. R. (2007). Mutation and citrullination modifies vimentin to a novel autoantigen for rheumatoid arthritis. Arthritis and rheumatism, 56(8), 2503-2511. doi.org/10.1002/art.22817
Kuna A. T. (2012). Mutated citrullinated vimentin antibodies in rheumatoid arthritis. Clinica chimica acta; international journal of clinical chemistry, 413(1-2), 66-73. doi.org/10.1016/j.cca.2011.10.020
AstriRasmussen, A., Ice, J. A., Li, H., Grundahl, K., Kelly, J. A., Radfar, L., Stone, D. U., Hefner, K. S., Anaya, J. M., Rohrer, M., Gopalakrishnan, R., Houston, G. D., Lewis, D. M., Chodosh, J., Harley, J. B., Hughes, P., Maier-Moore, J. S., Montgomery, C. G., Rhodus, N. L., Farris, A. D., Ä Sivils, K. L. (2014). Comparison of the American-European Consensus Group Sjogren's syndrome classification criteria to newly proposed American College of Rheumatology criteria in a large, carefully characterised sicca cohort. Annals of the rheumatic diseases, 73(1), 31-38. doi.org/10.1136/annrheumdis-2013-203845d
Shiboski, C. H., Shiboski, S. C., Seror, R., Criswell, L. A., Labetoulle, M., Lietman, T. M., Rasmussen, A., Scofield, H., Vitali, C., Bowman, S. J., Mariette, X., & International Sjogren's syndrome Criteria Working Group (2017). 2016 American College of Rheumatology/European League Against Rheumatism classification criteria for primary Sjogren's syndrome: A consensus and data-driven methodology involving three international patient cohorts. Annals of the rheumatic diseases, 76(1), 9-16. doi.org/10.1136/annrheumdis-2016-210571
Bagavant, H., Cizio, K., Araszkiewicz, A. M., Papinska, J. A., Garman, L., Li, C., Pezant, N., Drake, W. P., Montgomery, C. G., & Deshmukh, U. S. (2022). Systemic immune response to vimentin and granuloma formation in a model of pulmonary sarcoidosis. Journal of translational autoimmunity, 5, 100153. doi.org/10.1016/j.jtauto.2022.100153
Fayyaz, A., Kurien, B. T., & Scofield, R. H. (2016). Autoantibodies in Sjogren's syndrome. Rheumatic diseases clinics of North America, 42(3), 419-434. doi.org/10.1016/j.rdc.2016.03.002
Li, Y. H., Gao, Y. P., Dong, J., Shi, L. J., Sun, X. L., Li, R., Zhang, X. W., Liu, Y., Long, L., He, J., Zhong, Q. J., Morand, E., Yang, G., & Li, Z. G. (2018). Identification of a novel autoantibody against self-vimentin specific in secondary Sjogren's syndrome. Arthritis research & therapy, 20(1), 30. doi.org/10.1186/s13075-017-1508-5

What is claimed is:

1. An assay for detecting a biomarker comprising:
obtaining or having obtained a biological sample from the subject suffering from Sjögren's syndrome; and
assaying the biological sample obtained from the subject to determine an amount of anti-vimentin antibodies;
wherein the amount of anti-vimentin autoantibodies is detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection, wherein vimentin used in the assay is a post-translationally modified soluble vimentin; and
if the patient has a higher amount of anti-vimentin autoantibodies when compared to a healthy control or an average of patients with moderate to low symptoms of SS, identifying that the patient has a higher severity Sjögren's syndrome.

2. The assay of claim 1, wherein the biological sample is selected from a saliva, a blood, a plasma, a serum, or a tear sample.

3. The assay of claim 1, wherein the presence of anti-vimentin antibodies is indicative of a higher severity Sjögren's syndrome.

4. The assay of claim 3, wherein the higher severity Sjögren's syndrome is oral dryness, ocular dryness, or both.

5. A method of detecting and treating one or more symptoms of Sjögren's syndrome in a subject comprising:
obtaining or having obtained a biological sample from the subject;
detecting an amount of anti-vimentin antibodies in the biological sample, using as a capture agent a post-translationally modified soluble vimentin; and
administering one or more therapeutic compositions selected from the group consisting of saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome if the amount of anti-vimentin antibodies in the sample is higher than in a healthy control or an average of patients with moderate to low symptoms of Sjögren's syndrome.

6. The method of claim 5, wherein the anti-vimentin antibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

7. The method of claim 5, wherein the biological sample is selected from a saliva, a blood, a plasma, a serum, or a tear sample.

8. The method of claim 5, wherein the higher amount of anti-vimentin antibodies when compared to an amount in a healthy control or an average of patients with moderate to low symptoms of SS is indicative of a higher severity Sjögren's syndrome.

9. The method of claim 8, wherein the higher severity Sjögren's syndrome is oral dryness, ocular dryness, or both.

10. An assay for detecting a higher severity Sjögren's syndrome consisting essentially of:
assaying a biological sample obtained from the subject suffering from one or more symptoms of Sjögren's syndrome for an amount of anti-vimentin autoantibodies, wherein the biological sample is selected from the group consisting of tears, saliva, salivary gland tissue, blood, serum, and plasma;
applying an algorithm to the amount of anti-vimentin antibodies, the algorithm generating a Sjögren's syndrome score based on a comparison of the measured protein expression levels to reference levels;
wherein the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection, wherein vimentin used in the assay is a post-translationally modified soluble vimentin; and
wherein if the patient has a higher amount of anti-vimentin autoantibodies when compared to a healthy control or an average of patients with moderate to low symptoms of SS, identifying that the patient has a higher severity Sjögren's syndrome.

11. The assay of claim 10, wherein the biological sample is selected from a saliva, a blood, a plasma, a serum, or a tear sample.

12. The assay of claim 10, wherein the higher severity Sjögren's syndrome is oral dryness, ocular dryness, or both.

13. A kit comprising:
a synthetic or recombinant peptide or protein, covalently attached to a solid support or a detectable label, wherein the synthetic or recombinant peptide or protein is vimentin, wherein the vimentin is a post-translationally modified soluble vimentin; and
instructions for detecting an amount of anti-vimentin antibodies in a biological sample from a patient suspected of having Sjögren's syndrome and comparing to an amount of anti-vimentin antibodies in a healthy control or an average of patients with moderate to low symptoms of Sjögren's syndrome.

14. The kit of claim 13, wherein the solid support is selected from the group consisting of a multiwell plate, an enzyme-linked immunosorbent assay (ELISA) plate, a microarray, a bead, a porous strip, and a nitrocellulose filter.

15. The kit of claim 13, wherein the kit is an assay selected from the group consisting of a multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

16. The kit of claim 13, further comprising a secondary antibody labeled directly or indirectly with a detectable moiety.

17. The kit of claim 13, wherein if the amount of anti-vimentin antibodies in a sample is higher than an amount in a healthy control or an average of patients with moderate to low symptoms of SS, the higher amount of anti-vimentin antibodies is indicative of higher severity Sjögren's syndrome determined by oral and ocular dryness.

18. A method of detecting a biomarker comprising:
obtaining a biological sample from the patient suspected of having Sjögren's syndrome (SS);
detecting or having detected an amount of anti-vimentin autoantibodies in the biological sample using as a capture agent a post-translationally modified soluble vimentin;
comparing the amount of anti-vimentin autoantibodies in the biological sample to an amount in a healthy control or an average of patients with moderate to low symptoms of SS; and
determining, by a computer device, that the patient has a higher amount of anti-vimentin autoantibodies when compared to the healthy control or patients with moderate to low symptoms of SS; and if the patient has a higher amount of anti-vimentin autoantibodies identifying that the patient has a higher severity Sjögren's syndrome.

19. The method of claim 18, wherein the anti-vimentin autoantibodies are detected using an assay selected from at least one of: multiplex bead-based assay, capillary Western Blot, ELISA, flow cytometry, precipitin, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

20. The method of claim 18, wherein the biological sample is selected from a saliva, a blood, a plasma, a serum, or a tear sample.

21. The method of claim 18, further comprising the step of treating the patient with at least one of: saliva substitutes, saliva stimulants, interferon-alpha, artificial tears, methylcellulose inserts, eye ointments, muscarinic agonist drugs, immunomodulatory drugs, and autologous serum drops in an amount effective to treat one or more symptoms of Sjögren's syndrome in the subject having the higher severity Sjögren's syndrome.
